(19) United States
(12) Patent Application Publication
(10) Pub. No.: US 2012/0179080 A1
(43) Pub. Date: Jul. 12, 2012

(54) POSTURE-IMPROVING SUPPORT GARMENT

(75) Inventors: Akiko Sakamoto, Kyoto (JP); Mayumi Umemoto, Kyoto (JP); Mai Sudo, Kyoto (JP)

(73) Assignee: Wacoat Corp., Kyoto (JP)

(21) Appl. No.: 13/318,156

(22) PCT Filed: Apr. 28, 2010
(86) PCT No.: PCT/JP2010/057598

§ 371 Date: Dec. 6, 2011

(30) Foreign Application Priority Data
May 1, 2009 (JP) 2009-112004

(51) Int. Cl. A61F 5/00

(52) U.S. Cl. 602/19

(57) ABSTRACT

A posture-improving support garment according to an embodiment of the present invention is one comprising: a main body section 10 covering at least the upper body; and a beltlike straining section 20 having a straining force stronger than that of the main body section 10, wherein the straining section 20 extends right and left downward from at least any one of the seventh to ninth thoracic vertebrae, passes via lower portions of the right and left ribs, and joins at a center of the lower abdomen, and wherein at the center of the lower abdomen, a center line of the straining section 20 is located between the navel and the upper end of the pubis.

### TECHNICAL FIELD

The present invention relates to a garment supporting improvement in posture.

### BACKGROUND ART

Non Patent Literatures 1 to 3 describe that it is important to know/comprehend the position of the center of gravity of a human body in analysis of posture and movement of the body. In general, the position of the center of gravity is defined as a point where weights of respective sections of the body are at equilibrium with each other, and the position is believed to be located a little in front of the sacrum (i.e., near the second sacral vertebra) in the pelvis in the fundamental standing position. This position of the center of gravity of the body varies depending upon postures and was not easy to specify, but Non Patent Literatures 1 to 3 proved that when the body is divided into upper and lower bodies with respect to the position of the center of gravity of the body, the position of the center of gravity of the body can be readily specified by the midpoint between the centers of gravity of the upper body and the lower body. They describe that the center of gravity of the upper body is located around the seventh to ninth thoracic vertebrae, particularly at the seventh thoracic vertebra, and the center of gravity of the lower body is located between near half and upper one third of the femoral area.

On the other hand, Non Patent Literatures 4 to 6 focus attention on posture and movement of only the human upper body. These Non Patent Literatures proved that when the upper body is divided into upper and lower parts with respect to the seventh thoracic vertebra which is the position of the center of gravity of the upper body, the upper part and the lower part of the upper body rotate in opposite directions during walking, thereby to maintain a balance in the upper body. Namely, they proved that even in the case of the upper body only, the balance is maintained in the upper body with the seventh thoracic vertebra serving as a fulcrum.

With focus on this center of gravity of the upper body, i.e., the seventh to ninth thoracic vertebrae, Patent Literature 1 discloses the invention to facilitate prevention and treatment of disorders of the muscles at and near the shoulder joints in the upper part of the upper body, and the muscles of the arms near the shoulder joints. In the shoulder and arm support garment described in this Patent Literature 1, a base garment of stretchable fabric with at least an upper body section has strong-straining-force portions with a strong straining force in partial areas, and each of the strong-straining-force portions is provided so as to extend from an end-neighboring portion slightly shifted from the acromion toward the back center and located on the acromion side of the upper part of the trapezius muscle of the human body, via a region near the angulus superior scalpula and then via a region near any one of the seventh to ninth thoracic vertebrae, up to a region near the subcostal area on the opposite side.

### CITATION LIST

Patent Literature
Patent Literature 1: Japanese Patent No. 3115816

Non Patent Literatures
Non Patent Literature 1: Yuko Sato and three others, “Visual Assessment of Center of Gravity in Analysis of Posture and Movement,” Journal of the Japanese Physical Therapy Association, Vol. 23, special number (the thirty first meeting in Aichi), 1996, p. 176
Non Patent Literature 2: Yuko Sato and three others, “Visual Assessment of Center of Gravity in Analysis of Posture and Movement -second report-,” Journal of the Japanese Physical Therapy Association, Vol. 25, special number (the thirty third meeting in Kyoto), 1998, p. 158
Non Patent Literature 3: Yuko Kubo and three others, “Investigation of the Validity of Visual Assessment of Center of Gravity in the Analysis of Posture and Movement,” Journal of the Japanese Physical Therapy Association, Vol. 33, No. 3, 2006, p. 112-117
Non Patent Literature 4: Yuko Kubo and three others, “Movement of Upper Body during Walking Observed from Center of Gravity of Upper Body,” Journal of the Japanese Physical Therapy Association, Vol. 28, 2001, p. 177
Non Patent Literature 5: Yuko Kubo and three others, “Movement of Upper Body during Walking Observed from Center of Gravity of Upper Body (second report),” Journal of the Japanese Physical Therapy Association, Vol. 29, 2002, p. 161
Non Patent Literature 6: Yuko Kubo and three others, “Movement of Upper Body during Walking Observed from Center of Gravity of Upper Body (third report),” Journal of the Japanese Physical Therapy Association, Vol. 30, 2003, p. 152

### SUMMARY OF INVENTION

### Technical Problem

Incidentally, there are demands for a garment that, when simply worn, can support not only the upper part of the upper body but also the lower part of the upper body, i.e., the lower region below the seventh to ninth thoracic vertebrae. Specifically, there are demands for supporting improvement in posture, e.g., a hunchback and a bulge of the lower abdomen.

It is therefore an object of the present invention to provide a posture-improving support garment capable of supporting the improvement in posture, particularly the improvement of the hunchback and the bulge of the lower abdomen.

### Solution to Problem

The inventors focused attention on use of the seventh to ninth thoracic vertebrae as the center of gravity of the upper body in supporting the improvement of the posture such as the hunchback and the bulge of the lower abdomen, i.e., the seventh to ninth thoracic vertebrae with little positional variation as a fulcrum of rotations of the upper and lower parts of the upper body during movement, e.g., during walking.

A posture-improving support garment according to the present invention is a posture-improving support garment comprising: a main body section covering at least the upper body; and a beltlike straining section having a straining force stronger than that of the main body section, wherein the straining section extends right and left downward from at least any one of the seventh to ninth thoracic vertebrae, passes via lower portions of the right and left ribs, and joins at a center of the lower abdomen, and wherein at the center of the lower abdomen, a center line of the straining section is located between the navel and the upper end of the pubis.

There are the following two major reasons conceivable as causes of a bad posture. First, the erector spinae muscles become weak and the thoracic vertebrae become curved backward, resulting in a hunchback. Second, the abdominal muscles become weak to lower the abdominal muscle pressure and the lumbar vertebrae become curved forward, resulting in a bulge of the lower abdomen.

According to the present invention, the straining section attracts the seventh to ninth thoracic vertebrae and the center of the lower abdomen to each other and thus acts to support the erector spinae muscles and the abdominal muscles simultaneously and to work on the erector spinae muscles and the abdominal muscles simultaneously to stimulate muscle contraction. Particularly, it works on the abdominal muscles to increase the abdominal muscle pressure. As a consequence, it reduces the backward curvature of the thoracic vertebrae and the forward curvature of the lumbar vertebrae so as to straighten the back and tighten the lower abdomen.

Since the seventh to ninth thoracic vertebrae, which serve as a fulcrum of rotations of the upper and lower parts of the upper body during movement such as walking and have little positional variation, serve as a fulcrum, it is feasible to stably implement the support and work on the erector spinae muscles and the abdominal muscles.

The foregoing posture-improving support garment preferably further comprises a lumbar straining section which extends right and left from the center of the lower abdomen, passes via the ilia in the pelvis, and joins at the lower back.

This configuration allows the lumbar straining section to further enhance the abdominal muscle pressure of the lower abdomen. Moreover, since the lumbar straining section supports the lumbar region while surrounding it from the lower abdomen, the position of the pelvis is stabilized by increase of the abdominal muscle pressure. Furthermore, since the lumbar straining section supports the lumbar region while surrounding it from the lower abdomen, it reduces right-and-left and front-and-back positional deviation of the pelvis due to movement such as walking, so as to keep the position of the pelvis stabler.

The foregoing posture-improving support garment preferably further comprises a shoulder straining section which extends right and left upward from at least any one of the seventh to ninth thoracic vertebrae, and extends along the scapular spines in the shoulder blades.

This configuration allows the shoulder straining section to support the shoulders along the scapular spines of the shoulder blades, whereby the shoulder blades are adducted so as to spread the shoulders or so as to stick out the chest. Therefore, the garment can provide greater support for the posture improvement.

Preferably, at least one of a portion of the straining section passing via the seventh to ninth thoracic vertebrae and a portion of the straining section passing via the center of the lower abdomen has a straining force stronger than that of the other portion of the straining section.

As described above, in order to achieve the improvement in posture such as the hunchback and the bulge of the lower abdomen, it is rational to attract starting points at the seventh to ninth thoracic vertebrae as the center of gravity of the upper body and at the region near the center of the lower abdomen as the most projecting portion in the abdominal region, to each other. In this configuration, since the straining section imparts the stronger straining force on the region of the seventh to ninth thoracic vertebrae and the central region of the lower abdomen where the maximum attractive force is applied, than that on the other portion, stability is provided in the attraction between the seventh to ninth thoracic vertebrae and the center of the lower abdomen to each other.

A width of a dorsal portion and a width of a lateral portion of the straining section are not less than 4 cm and not more than 7 cm.

### Advantageous Effects of Invention

The present invention enables the support for the posture improvement, particularly the improvement of the hunchback and the bulge of the lower abdomen.

### BRIEF DESCRIPTION OF DRAWINGS

FIG. 1 is a perspective view showing front appearance of a posture-improving support garment according to an embodiment of the present invention.

FIG. 2 is a drawing showing back appearance of the posture-improving support garment according to the embodiment of the present invention.

FIG. 3 is a drawing showing side appearance of the posture-improving support garment according to the embodiment of the present invention.

FIG. 4 is a drawing showing a front view of the human skeletal structure.

FIG. 5 is a drawing showing a back view of the human skeletal structure.

FIG. 6 is a drawing showing a side view of the human skeletal structure.

FIG. 7 is a drawing showing the structure of human erector spinae muscles.

FIG. 8 is a drawing showing the structure of human abdominal muscles.

FIG. 9 is a drawing showing side views of states of human vertebrae with and without the posture-improving support garment of the embodiment.

FIG. 10 is a drawing showing front and back appearance of the posture-improving support garment according to a modification example of the present invention.

FIG. 11 is a drawing showing front and back appearance of the posture-improving support garment according to another modification example of the present invention.

FIG. 12 is a drawing showing front and back appearance of the posture-improving support garment according to still another modification example of the present invention.

### DESCRIPTION OF EMBODIMENTS

The preferred embodiments of the present invention will be described below in detail with reference to the drawings. In the drawings identical or equivalent portions will be denoted by the same reference signs.

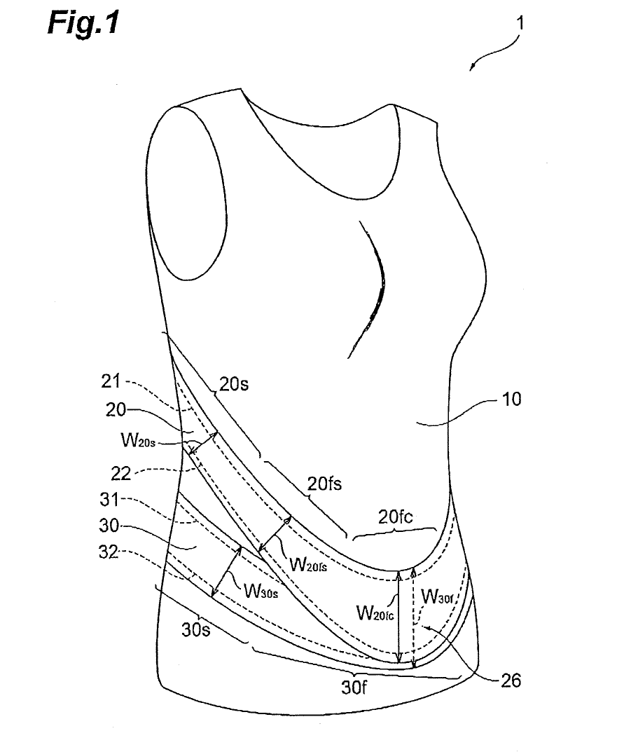

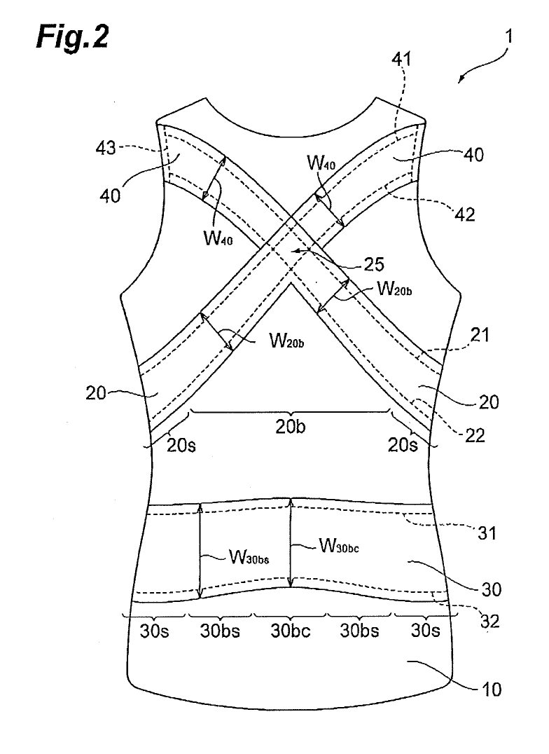

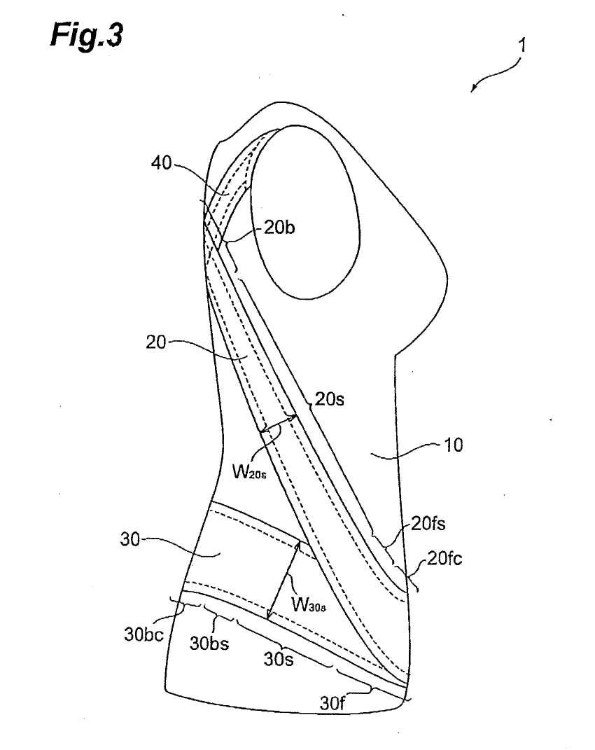

FIG. 1 is a perspective view showing front appearance of the posture-improving support garment according to an embodiment of the present invention, and FIG. 2 is a drawing showing back appearance of the posture-improving support garment according to the embodiment of the present invention. Furthermore, FIG. 3 is a drawing showing side appearance of the posture-improving support garment according to the embodiment of the present invention. The posture-improving support garment 1 shown in FIGS. 1 to 3 is provided with a main body section 10, a main straining section 20, a lumbar straining section 30, and a shoulder straining section 40.

The main body section 10 is a garment of a sleeveless type covering the upper body and lower abdomen of a wearer. The main body section 10 is made of stretch cloth and has a tight fitting property with the wearer's body. The main body section 10 is provided with the main straining section 20, the lumbar straining section 30, and the shoulder straining section 40 in partial areas.

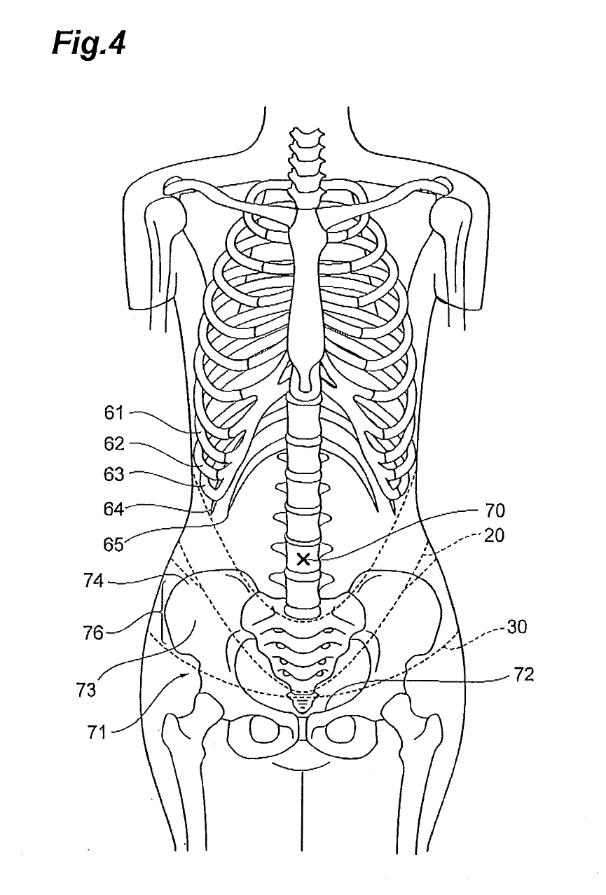

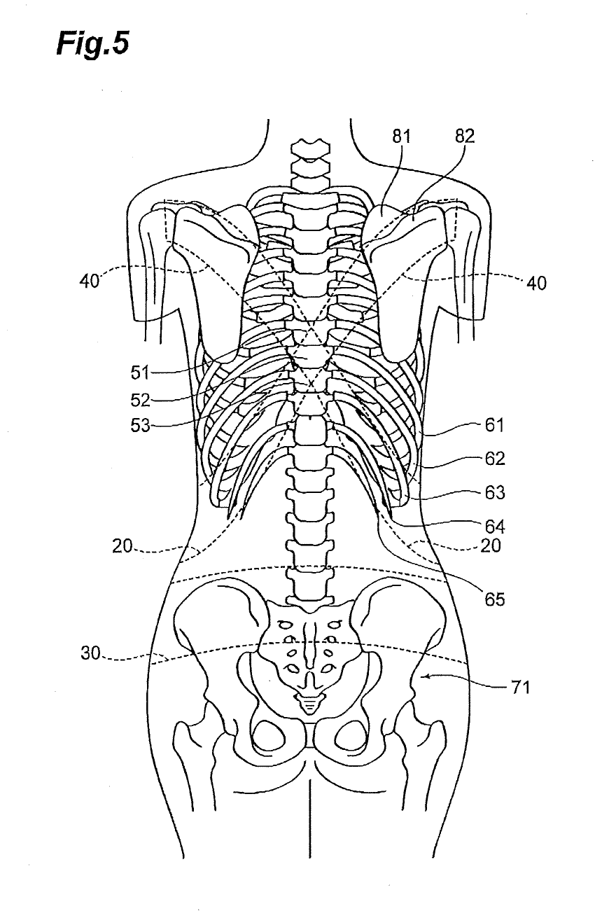

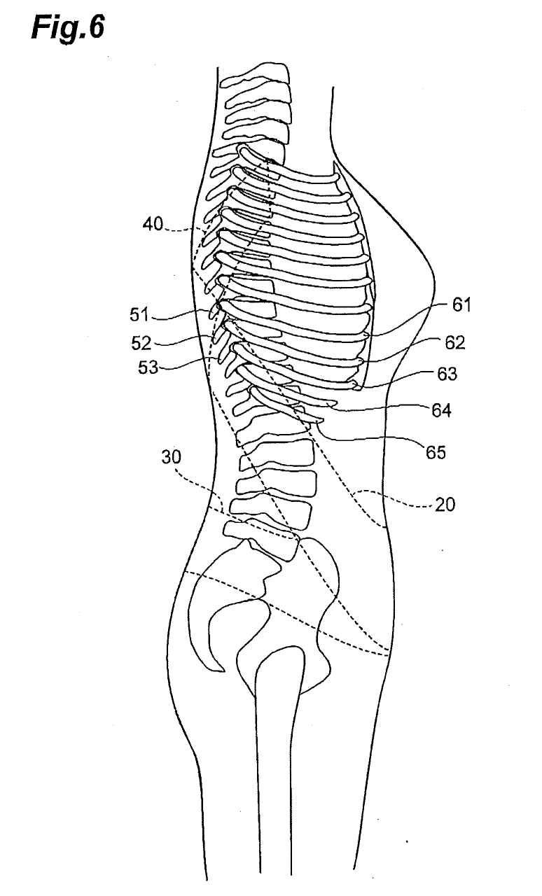

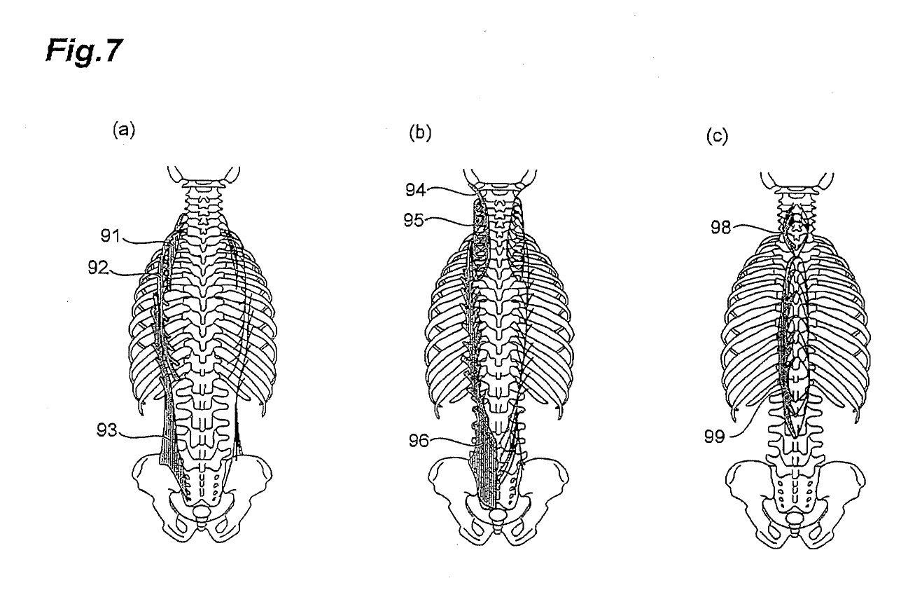

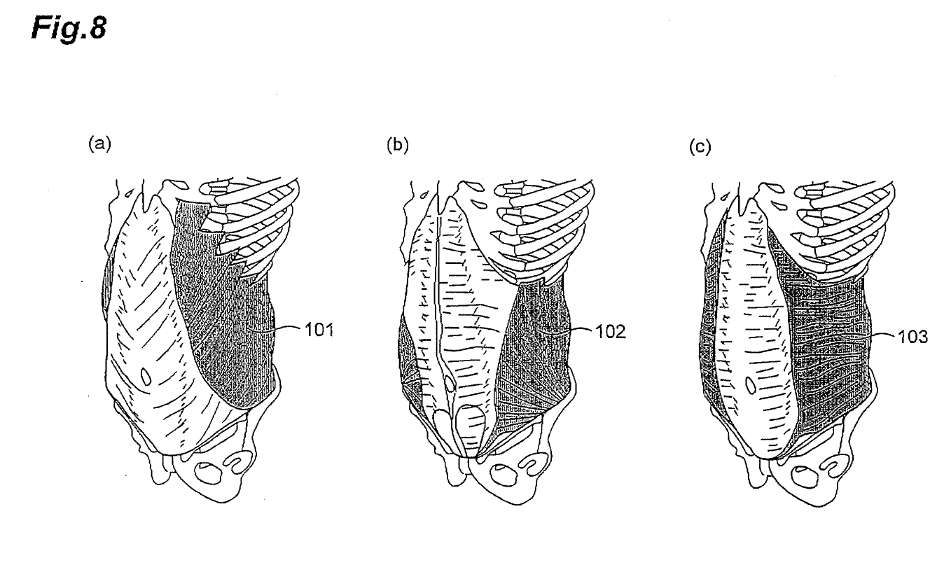

The below will describe the main straining section 20, the lumbar straining section 30, and the shoulder straining section 40, with as-needed reference to the front view, back view, and side view of the human skeletal structure shown in FIGS. 4 to 6, the structure figure of the human erector spinae muscles shown in FIG. 7, and the structure figure of the human abdominal muscles shown in FIG. 8.

Each of the main straining section 20, the lumbar straining section 30, and the shoulder straining section 40 is of a beltlike shape with a straining force stronger than that of the main body section 10. A highly stretchable fabric such as Powernet is applicable to the main straining section 20, the lumbar straining section 30, and the shoulder straining section 40. The main straining section 20 is sewn to the main body section 10 at widthwise sides 21, 22. Similarly, the lumbar straining section 30 is also sewn to the main body section 10 at widthwise sides 31, 32 and the shoulder straining section 40 is also sewn to the main body section 10 at widthwise sides 41, 42 and longitudinal ends 43.

The main straining section 20, in a worn state, extends in an inverted V-shape right and left downward from the center of the thoracic vertebrae at the wearer's back, i.e., from at least any one of the seventh to ninth thoracic vertebrae 51-53 shown in FIGS. 4 to 6. The main straining section 20, in the worn state, then extends forwardly downward of the wearer along tips on the wearer's front side of the lower parts of the right and left ribs at the wearer's sides, i.e., at least any of the false ribs (eighth to twelfth ribs) 61-65 or the floating ribs (eleventh and twelfth ribs) 64, 65. Next, the main straining section 20, in the worn state, joins at the center of the lower abdomen on the wearer's front side, i.e., in a region from near the navel 70 to near the upper end 72 of the pubis in the pelvis 71. At the center of the lower abdomen, the main straining section 20 is located so that a center line along the width direction (or the vertical direction) of the main straining section 20 is positioned between the navel 70 and the pubis upper end 72. It is noted that parts of the main straining section 20 may cover the navel 70 and the pubis upper end 72.

In this manner, the main straining section 20 acts to attract the seventh to ninth thoracic vertebrae 51-53 and the center of the lower abdomen to each other with the seventh to ninth thoracic vertebrae 51-53 serving as a fulcrum. As a consequence, the main straining section 20 acts to support the erector spinae muscles consisting of the iliocostal muscles shown in FIG. 7 (a), the longissimus muscles shown in FIG. 7 (b), and the spinal muscles 97-99 shown in FIG. 7 (c), i.e., the erector spinae muscles consisting of the musculus iliocostalis cervicis 91, the musculus iliocostalis thoracis 92, the musculus iliocostalis lumborum 93, the musculus longissimus capitis 94, the musculus longissimus cervicis 95, the musculus longissimus thoracis 96, the musculus spinalis capitis 97 (not shown), the musculus spinalis cervicis 98, and the musculus spinalis thoracis 99, and to work on these erector spinae muscles to stimulate muscle contraction.

The main straining section 20 also acts to support the abdominal muscles consisting of the external abdominal oblique muscles 101 extending from the tips of the false ribs 61-65 or the floating ribs 64, 65 to the center of the lower abdomen as shown in FIG. 8 (a), the internal abdominal oblique muscles 102 located inside the external abdominal oblique muscles 101 as shown in FIG. 8 (b), and the transverse abdominal muscles 103 located inside the internal abdominal oblique muscles 102 as shown in FIG. 8 (c), and to work on these abdominal muscles to stimulate muscle contraction. Particularly, the main straining section 20 is located along muscle fibers of the external abdominal oblique muscles 101 extending from the tips of the false ribs 61-65 or the floating ribs 64, 65 to the center of the lower abdomen. Since a muscle contracts in its muscle fiber direction, the main straining section 20 works on the external abdominal oblique muscles 101 stronger.

Next, the lumbar straining section 30, in the worn state, extends right and left from the center of the lower abdomen on the wearer's front side, i.e., from near the region between the navel 70 and the pubis upper end 72 in the pelvis 71. Then the lumbar straining section 30, in the worn state, extends to the wearer's back via the outer lip sides 76 of the iliac crests 74 of the ilia 73 of the pelvis 71 at the wearer's sides. Subsequently, the lumbar straining section 30, in the worn state, joins near the lumbar at the wearer's back, i.e., near the upper parts of the ilia 73 in the pelvis 71.

In this manner, the lumbar straining section 30 acts to additionally support the abdominal muscles shown in FIG. 8 and to additionally work on these abdominal muscles to stimulate muscle contraction. Furthermore, the lumbar straining section 30 acts to support the lumbar region while surrounding it from the lower abdomen, so as to stabilize the position of the pelvis 71.

Next, the shoulder straining section 40, in the worn state, extends right and left upward from the center of the thoracic vertebrae at the wearer's back, i.e., from at least any of the seventh to ninth thoracic vertebrae 51-53 shown in FIGS. 4 to 6. Then the shoulder straining section 40, in the worn state, extends along the longitudinal directions of the scapular spines 82 in the shoulder blades 81. Namely, it extends to the right and left along the scapular spines 82 stretching approximately along the horizontal direction.

In this manner, the shoulder straining section 40 acts to adduct the shoulder blades 81 so as to spread the shoulders or so as to stick out the chest.

Now, the width W_{20b} of back portion 20b and the width W₂₀ₛ of side portions 20s in the main straining section 20 can be not less than about 4 cm and not more than about 7 cm and are preferably not less than about 4 cm and not more than about 6 cm. The width W_{20b} of back portion 20b and the width W₂₀ₛ of side portions 20s are most preferably about 5 cm. The grounds for it will be explained in Examples below. On the other hand, the width W_{20fc} of front center portion 20fc in the main straining section 20 is preferably not less than about 7 cm and not more than about 14 cm. For example, the width W_{20fc} of front center portion 20fc is most preferably about 8 cm for women and about 14 cm for men. This configuration allows the main straining section 20 to act to support the abdominal muscles over the entire lower abdomen and to work on these abdominal muscles to stimulate muscle contraction. The width W_{20fs} of each front side portion 20fc in the main straining section 20 gradually increases from the side portion 20s to the front center portion 20fc. It is noted herein that the widths W_{20b}, W₂₀ₛ, W_{20fc}, and W_{20fs} of the main straining section 20 are side-side distances along a straight line nearly perpendicular to the side edges of the main straining section 20.

On the other hand, the width W_{30f} of lower abdominal portion 30f and the width W₃₀ₛ of side portions 30s in the lumbar straining section 30 are preferably not less than about 7 cm and not more than about 14 cm. For example, the width W_{30f} of lower abdominal portion 30f and the width W₃₀ₛ of side portions 30s are most preferably about 8 cm for women and about 14 cm for men. The width W_{30bc} of back center portion 30bc in the lumbar straining section 30 is not less than about 5 cm and is preferably about 6 cm. For example, the width W_{30bc} of back center portion 30bc is most preferably about 6 cm for women and about 9 cm for men. The width W_{30bs} of back side portions 30bs in the lumbar straining section 30 gradually increases from the back center portion 30bc to the side portions 30s. This configuration allows the lumbar straining section 30 to firmly support the pelvis 71, i.e., the lower abdomen and the lumbar region. It is noted herein that the widths W_{30f}, W₃₀ₛ, W_{30bc}, and W_{30bs} of the lumbar straining section 30 are side-side distances along a straight line nearly perpendicular to the side edges of the lumbar straining section 30.

In the present embodiment, the central region of the lower abdominal portion of the lumbar straining section 30 is sewn to the main body section 10 while overlapping the front center portion 20fc and front side portions 20fs in the main straining section 20. The lumbar straining section 30 may be made of a single fabric so as to be integral with the main straining section 20.

The width W₄₀ of the shoulder straining section 40 may be identical to the width W_{20b} of the back portion 20b and the width W₂₀ₛ of the side portions 20s in the main straining section 20. This allows the main straining section 20 and the shoulder straining section 40 to be integrally made of a single fabric. It is noted herein that the width W₄₀ of the shoulder straining section 40 is a side-side distance along a straight line nearly perpendicular to the side edges of the shoulder straining section 40.

In the present embodiment, the main straining section 20 extending right and left downward from the seventh to ninth thoracic vertebrae 51-53 is sewn with an overlap in part 25 at the seventh to ninth thoracic vertebrae 51-53. Furthermore, as described above, the front center portion 20fc and front side portions 20fs of the main straining section 20 and the central region of the lower abdominal portion of the lumbar straining section 30 are sewn with an overlap in part 26 at the center of the lower abdomen. This makes straining forces at the part 25 passing via the seventh to ninth thoracic vertebrae 51-53 and at the part 26 passing via the center of the lower abdomen, stronger than those at the other parts in the main straining section 20, the lumbar straining section 30, and the shoulder straining section 40. For making the straining forces stronger at the part 25 passing via the seventh to ninth thoracic vertebrae 51-53 and at the part 26 passing via the center of the lower abdomen, it can be contemplated that the elasticity of the fabric for the portions passing the seventh to ninth thoracic vertebrae 51-53 and the portions passing the center of the lower abdomen in the main straining section 20, lumbar straining section 30, and shoulder straining section 40 is made different from that of the fabric for the other portions.

Incidentally, a man of a bad posture is conceivably characterized by the following points.

forward inclination of the neck

hunchback (round shoulders)

bulge of the abdomen

Conceivable reasons for the bad posture are as follows:

weak abdominal muscles (external abdominal oblique muscles, internal abdominal oblique muscles, and transverse abdominal muscles);

low abdominal muscle pressure;

descent of the diaphragm;

failure in firm support of the backbone.

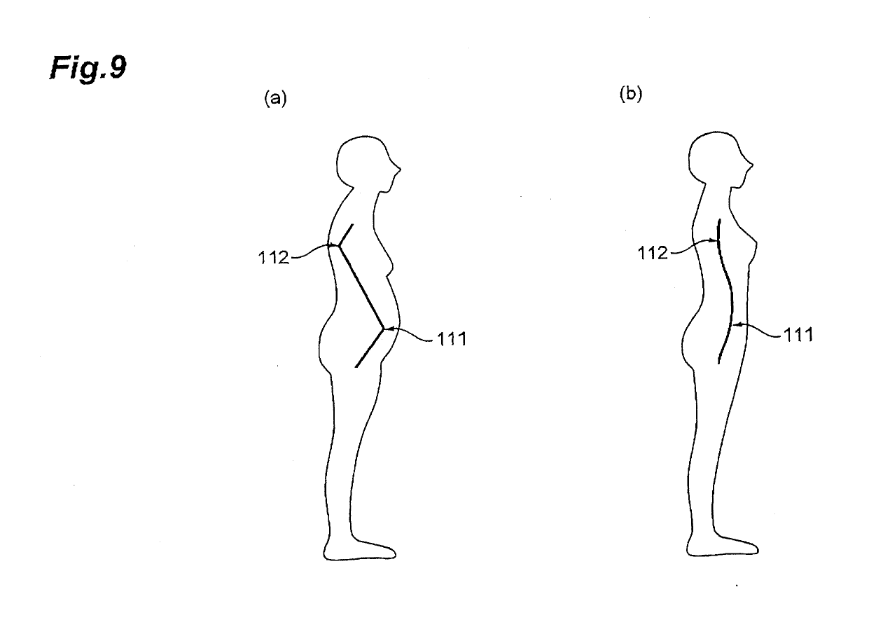

These result in, as shown in FIG. 9 (a),

forward curvature of the lumbar vertebrae 111, and

backward curvature of the thoracic vertebrae 112.

In the posture-improving support garment 1 of the present embodiment, since the main straining section 20 attracts the seventh to ninth thoracic vertebrae 51-53 and the center of the lower abdomen to each other, it acts to simultaneously support the erector spinae muscles and the abdominal muscles and to simultaneously work on the erector spinae muscles and the abdominal muscles to stimulate muscle contraction. Particularly, it works on the abdominal muscles to increase the abdominal muscle pressure. As a consequence, as shown in FIG. 9 (b), the posture-improving support garment reduces the forward curvature of the lumbar vertebrae 111 and the backward curvature of the thoracic vertebrae 112, so as to expand the thorax, stretch the back, and tighten the lower abdomen.

Since the fulcrum is located at the seventh to ninth thoracic vertebrae with little positional variation which serve as a fulcrum of rotations of the upper part and the lower part of the upper body during movement such as walking, the posture-improving support garment provides the stable support and work on the erector spinae muscles and the abdominal muscles even during movement such as walking. Furthermore, the garment also achieves a stabilizing effect to return the posture to a proper posture while the seventh to ninth thoracic vertebrae with little positional variation serve as a fulcrum.

Furthermore, in the posture-improving support garment 1 of the present embodiment, the lumbar straining section 30 further enhances the abdominal pressure of the lower abdomen. Since the lumbar straining section 30 supports the lumbar region while surrounding it from the lower abdomen, it increases the abdominal pressure to stabilize the position of the pelvis 71. Moreover, since the lumbar straining section 30 supports the lumbar region while surrounding it from the lower abdomen, it reduces right-and-left and front-and-back positional deviation of the pelvis 71 due to movement such as walking, so as to further stabilize the position of the pelvis 71. As a result, it is feasible to maintain the posture in a balanced manner.

Furthermore, in the posture-improving support garment 1 of the present embodiment, the shoulder straining section 40 supports the shoulders along the scapular spines 82 of the shoulder blades 81 to adduct the shoulder blades 81 so as to spread the shoulders or so as to stick out the chest. Therefore, it is feasible to better support the posture improvement.

As described above, in order to achieve the improvement in posture such as the hunchback and the bulge of the lower abdomen, it is rational to attract starting points at the seventh to ninth thoracic vertebrae 51-53 as the center of gravity of the upper body and at the region near the center of the lower abdomen as the most projecting portion in the abdominal region, to each other. In the posture-improving support garment 1 of the present embodiment, since the straining forces at the part 25 passing the seventh to ninth thoracic vertebrae 51-53 and at the part 26 passing the center of the lower abdomen are stronger than those at the other portions in the main straining section 20, lumbar straining section 30, and shoulder straining section 40, i.e., since the straining forces at the part 25 of the seventh to ninth thoracic vertebrae and at the central part 26 of the lower abdomen to which the maximum attractive force is applied are stronger than those at the other portions, it is feasible to achieve the stabilizing effect in the attraction between the seventh to ninth thoracic vertebrae 51-53 and the center of the lower abdomen to each other.

It should be noted that the present invention can be modified in many ways without being limited to the present embodiment as describe above. For example, the embodiment showed the example of the posture-improving support garment 1 with the main straining section 20, lumbar straining section 30, and shoulder straining section 40, but the posture-improving support garment can achieve the posture improving effect to straighten the back and tighten the lower abdomen as long as it is provided with at least the main straining section 20.

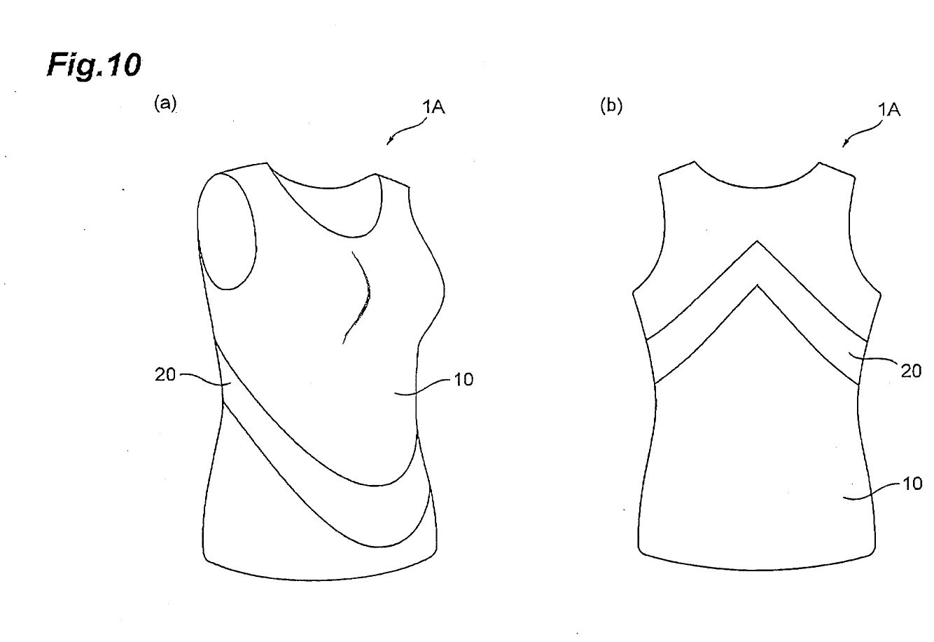

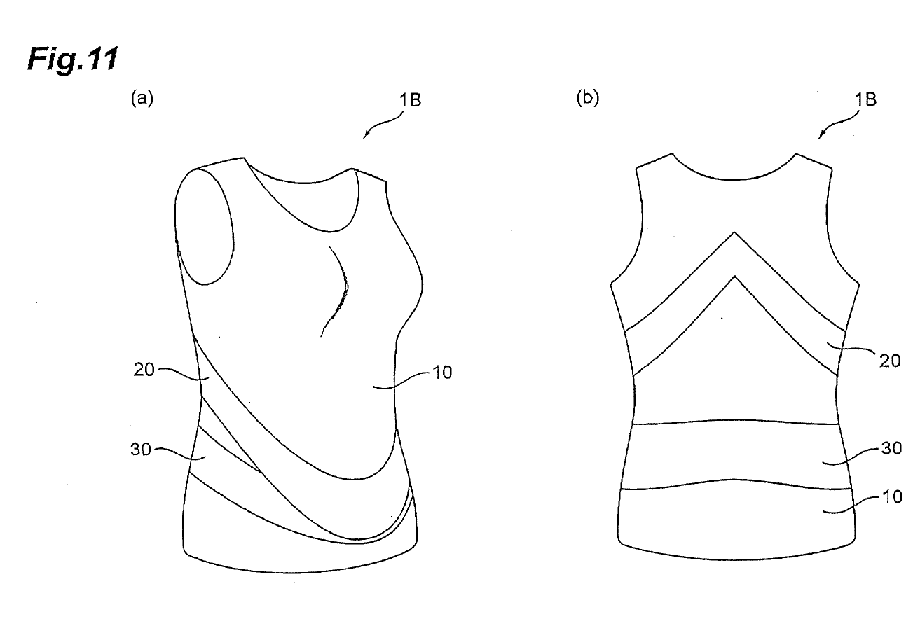

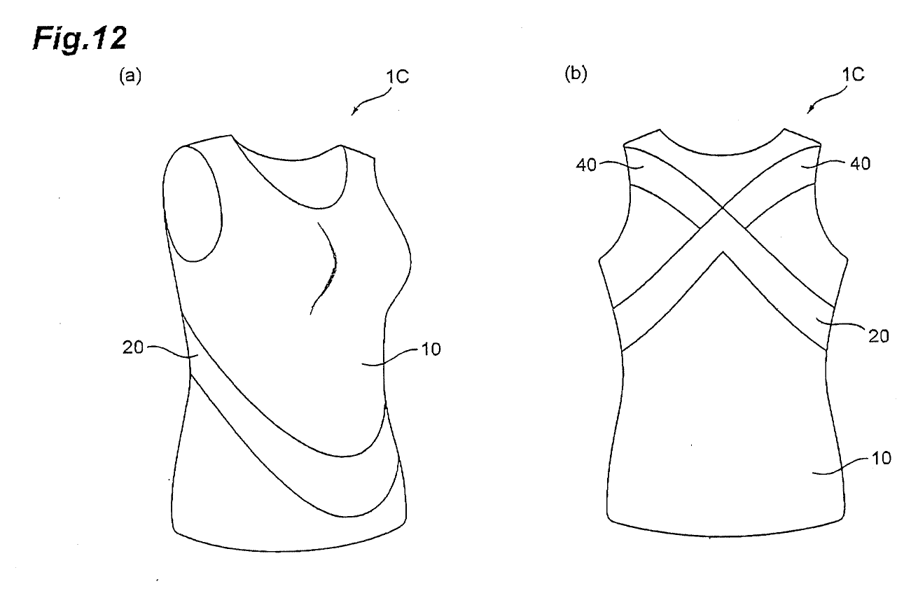

Specifically, the posture-improving support garment may be a form with the main straining section 20 only like posture-improving support garment 1A of modification example 1 shown in FIG. 10, or a form with the main straining section 20 and the lumbar straining section 30 like posture-improving support garment 1B of modification example 2 shown in FIG. 11, or a form with the main straining section 20 and the shoulder straining section 40 like posture-improving support garment 1C of modification example 3 shown in FIG. 12. In each of FIGS. 10 to 12, (a) shows a perspective view of the front side of the posture-improving support garment and (b) a back view of the posture-improving support garment.

The present embodiment was the example of sewing of the highly stretchable material (patch) for the main straining section 20, lumbar straining section 30, and shoulder straining section 40, but it is also possible to adopt a configuration wherein they are bonded by bonding or the like of the highly stretchable material. The main straining section 20, lumbar straining section 30, and shoulder straining section 40 may also be formed by change of knit systems (warp knitting, circular knitting, etc.) in formation of the main body section 10. The main straining section 20, lumbar straining section 30, and shoulder straining section 40 may also be formed by opal finish, resin print, specially-treated print, and so on. When the main straining section 20, lumbar straining section 30, and shoulder straining section 40 are formed through the use of the change of knit systems (warp knitting, circular knitting, etc.), opal finish, print, and so on as described above, they can be integrally made so that borders between them cannot be visually recognized.

The present embodiment showed the example of the posture-improving support garment of the sleeveless type, but the main body section in the posture-improving support garment may be one with sleeves, either long sleeves or short sleeves, or may be of a bodysuit type with a crotch part and leg parts.

### Examples 1 to 3

The posture-improving support garments 1 of the embodiment of the present invention shown in FIGS. 1 to 3 were produced as Examples 1 to 3 and evaluated by four female monitors. Examples 1 to 3 were different in the widths of the back part of the main straining section 20 and the shoulder straining section 40 and evaluated in contrast with each other. Since some width is necessary for support on the lower abdomen and the lumbar region, the width of the front center portion 20rc in the main straining section 20 and the widths of the lumbar straining section 30 were fixed as described below.

(Width W_{20fc} of Front Center Portion 20fc in Main Straining Section 20)

Examples 1 to 3: about 8 cm

(Width W₃₀₁ of Lower Abdominal Portion 30f and Width W₃₀ₛ of Side Portions 30s in Lumbar Straining Section 30)

Examples 1 to 3: about 8 cm

(Width W_{30bc} of Back Center Portion 30bc in Lumbar Straining Section 30)

Examples 1 to 3: about 6 cm

(Width W_{20b} of Back Portion 20b and Width W₂₀ₛ of Side Portions 20s in Main Straining Section 20, and Width W₄₀ of Shoulder Straining Section 40)

Example 1: about 5 cm
Example 2: about 7 cm, 1 cm wider on each side than Example 1
Example 3: about 3 cm, 1 cm narrower on each side than Example

In this evaluation, each monitor tried Examples 1 to 3 on and walked, and thereafter was requested for comment. According to the evaluation result, three out of the four monitors felt the best balance between the front and back straining forces to straighten the back and tighten the lower abdomen with the widths W_{20b}=W₂₀ₛ=W₄₀=5 cm. On the other hand, with the widths W_{20b}=W₂₀ₛ=W₄₀=7 cm, all the four monitors felt that the front and back straining forces were poorly balanced. As a result, they felt that the back straining force was too strong and the straining force on the lower abdomen was too weak, feeling insufficient tightening of the lower abdomen. With the widths W_{20b}=W₂₀ₛ=W₄₀=3 cm, one out of the four monitors felt optimum, whereas the three others felt that the front and back straining forces were poorly balanced. As a result, they felt that the back was unsteady, feeling no posture-improving support from the back.

It was thus verified from the above that the most preferred width W_{20b} of the back portion 20b in the main straining section 20 was 5 cm. It was also found that when the width W_{20b} of the back portion 20b in the main straining section 20 was expanded to 7 cm, the front and back straining forces became poorly balanced to result in feeling the back straining force too strong and the straining force on the lower abdomen too weak and it was difficult to improve the posture in a balanced manner. It was further found that when the width W_{20b} of the back portion 20b in the main straining section 20 was decreased to 3 cm, the front and back straining forces became poorly balanced to result in feeling the back straining force too weak and the straining force on the lower abdomen too strong and it was difficult to improve the posture in a balanced manner. It is thus expected from this result that the width W_{20b} of the back portion 20b in the main straining section 20 is preferably approximately in the range of 4 cm to 6 cm.

It is, however, expected that for men with relatively large back and strong muscular strength, the width W_{20b} of the back portion 20b in the main straining section 20 may be expanded to 7 cm, and then it is expected that the width W_{20b} of the back portion 20b in the main straining section 20 may be approximately in the range of 4 cm to 7 cm in view of unisex use.

### Example 1

Next, equivalents of above Example 1 as the posture-improving support garment 1 of the embodiment of the present invention as shown in FIGS. 1 to 3 were produced as Example 4 and evaluated by eleven female monitors. In this evaluation, Example 4 was evaluated by comparison with a conventional product.

The conventional product for comparison with Example 4 is a commercial product of the applicant and major product of the posture-improving support garment.

In this evaluation, each monitor was asked which she felt superior, by comparison between Example 4 of the present invention and the conventional product. According to the evaluation result, ten out of the eleven monitors felt that the posture was improved more with Example 4 than with the conventional product. Among them, seven monitors felt strengthening of the back and nine monitors felt tightening of the abdomen.

Furthermore, surprisingly, ten out of the eleven monitors answered that they felt less tight with Example 4 than with the conventional product. Nine out of the eleven monitors felt better mobility and improvement in posture during walking with Example 4 than with the conventional product. Particularly, eight out of the eleven monitors felt comfortable with Example 4 as a whole.

It was confirmed by the above result that Example 4 of the present invention was able to support the posture improvement, without degradation of comfort, e.g., tightness. Furthermore, it was also confirmed that even during movement such as walking, the posture improvement was stably supported without degradation of mobility. This is presumably because the fulcrum is defined at the seventh to ninth thoracic vertebrae with little positional variation serving as a fulcrum of rotations of the upper part and the lower part of the upper body during movement such as walking.

### INDUSTRIAL APPLICABILITY

The present invention is applicable to usage to support the posture improvement, particularly the improvement of the hunchback and the bulge of the lower abdomen.

### LIST OF REFERENCE SIGNS

1, 1A, 1B, 1C posture-improving support garment
10 main body section
20 main straining section (straining section)
20fc front center portion of main straining section
20fs front side portion of main straining section
20s side portion of main straining section
20b back portion of main straining section
25 part at seventh to ninth thoracic vertebrae
26 part passing via center of lower abdomen
30 lumbar straining section
30f lower abdominal portion of lumbar straining section
30s side portion of lumbar straining section
30bs back side portion of lumbar straining section
30bc back center portion of lumbar straining section
40 shoulder straining section
51-53 seventh to ninth thoracic vertebrae
61-65 false ribs (eighth to twelfth ribs)
64, 65 floating ribs (eleventh and twelfth ribs)
70 navel
71 pelvis
72 upper end of pubis
73 ilium
74 iliac crest
76 side of outer lip
81 shoulder blade
82 scapular spine
91-99 erector spinae muscles
91-93 iliocostal muscles
91 musculus iliocostalis cervicis
92 musculus iliocostalis thoracis
93 musculus iliocostalis lumborum
94-96 longissimus muscles
94 musculus longissimus capitis
95 musculus longissimus cervicis
96 musculus longissimus thoracis
97-99 spinal muscles
97 musculus spinalis capitis
98 musculus spinalis cervicis
99 musculus spinalis thoracis
101-103 abdominal muscles
101 external abdominal oblique muscle
102 internal abdominal oblique muscle
103 transverse abdominal muscle
111 lumbar vertebrae
112 thoracic vertebrae

1. A posture-improving support garment comprising:
a main body section covering at least the upper body; and a beltlike straining section having a straining force stronger than that of the main body section,
wherein the straining section extends right and left downward from at least any one of the seventh to ninth thoracic vertebrae, passes via lower portions of the right and left ribs, and joins at the center of the lower abdomen, and
wherein at the center of the lower abdomen, a center line of the straining section is located between the navel and the upper end of the pubis.

2. The posture-improving support garment according to claim 1, further comprising a lumbar straining section which extends right and left from the center of the lower abdomen, passes via the ilia in the pelvis, and joins at the lower back.

3. The posture-improving support garment according to claim 1, further comprising a shoulder straining section which extends right and left upward from at least any one of the seventh to ninth thoracic vertebrae, and extends along the scapular spines in the shoulder blades.

4. The posture-improving support garment according to claim 1, wherein at least one of a portion of the straining section passing via the seventh to ninth thoracic vertebrae and a portion of the straining section passing via the center of the lower abdomen has a straining force stronger than that of the other portion of the straining section.

5. The posture-improving support garment according to claim 1, wherein a width of a dorsal portion and a width of a lateral portion of the straining section are not less than 4 cm and not more than 7 cm.

6. The posture-improving support garment according to claim 3, wherein at least one of a portion of the straining section passing via the seventh to ninth thoracic vertebrae and a portion of the straining section passing via the center of the lower abdomen has a straining force stronger than that of the other portion of the straining section.

7. The posture-improving support garment according to claim 2, further comprising a shoulder straining section which extends right and left upward from at least any one of the seventh to ninth thoracic vertebrae, and extends along the scapular spines in the shoulder blades.

8. The posture-improving support garment according to claim 2, wherein at least one of a portion of the straining section passing via the seventh to ninth thoracic vertebrae and a portion of the straining section passing via the center of the lower abdomen has a straining force stronger than that of the other portion of the straining section.

9. The posture-improving support garment according to claim 7, wherein at least one of a portion of the straining section passing via the seventh to ninth thoracic vertebrae and a portion of the straining section passing via the center of the lower abdomen has a straining force stronger than that of the other portion of the straining section.
